(19) United States
(12) Patent Application Publication
(10) Pub. No.: US 2007/0090302 A1
(43) Pub. Date: Apr. 26, 2007

(54) Display device and fabricating method thereof

(75) Inventors: Seung-Hyun Son, Suwon-si (KR); Sung-Soo Kim, Suwon-si (KR)

Correspondence Address: STEIN, MCEWEN & BUI, LLP, 1400 EYE STREET, NW, SUITE 300, WASHINGTON, DC, 20005, US

(73) Assignee: Samsung SDI Co., Ltd., Suwon-si (KR)

(21) Appl. No.: 11/542,136

(22) Filed: Oct. 4, 2006

(30) Foreign Application Priority Data
Oct. 12, 2005 (KR) 2005-96232

(51) Int. Cl. A61N 5/00; G21G 5/00; H01L 31/00; G02F 1/136

(52) U.S. Cl. 250/492.2; 257/443; 349/42

(57) ABSTRACT

A display device capable of implementing a high resolution and a method of fabricating the same. The display device includes a substrate, a silicon member, an electron emission member, and a phosphor layer. The silicon member is attached to the substrate. The silicon member has a groove formed on at least a portion of an inner surface of the silicon member and forms a light-emitting space in cooperation with the substrate. The groove can be formed by chemical etch or deep reactive ion etching (DRIE) of a silicon wafer or a silicon on insulator (SOI) wafer. The electron emission member is disposed on a portion of the groove. The phosphor layer is disposed in the light-emitting space. The display device may further have an anode electrode disposed on the substrate. Such a display device is versatile and simple in structure and fabrication.

### CROSS-REFERENCE TO RELATED APPLICATIONS

This application claims the benefit of Korean Application No. 2005-96232, filed Oct. 12, 2005, in the Korean Intellectual Property Office, the disclosure of which is incorporated herein by reference.

### BACKGROUND OF THE INVENTION

1. Field of the Invention

Aspects of the present invention relate to a display device, and more particularly, to a display device capable of implementing high resolution while having a simple and efficient structure and a method of fabricating the same.

2. Description of the Related Art

A plasma display panel (PDP) is a flat panel device in which excitation gas is hermetically filled between two substrates on which a plurality of electrodes are formed. When a discharge voltage is applied across the electrodes, ultraviolet rays are generated to excite a phosphor formed in a predetermined pattern. The excited phosphor emits visible rays, thereby creating an image.

FIG. 1 is an exploded perspective view of a conventional alternating current (AC) PDP. Referring to FIG. 1, a PDP 10 includes a transparent front substrate 11 and a rear substrate 12. Stripe-shaped sustain electrodes 13a and bus electrodes 13c are formed on the front substrate 11, and a dielectric layer 14 and a protection layer 15 are formed on the resulting structure. Stripe-shaped address electrodes 13b, a dielectric layer 16, barrier ribs 17, and a phosphor layer 18 are formed on the rear substrate 12. The sustain electrodes 13a are perpendicular to the address electrodes 13b, and a discharge space is defined by the barrier ribs 17 to form a discharge cell.

However, the conventional PDP is complex in structure because it requires many processes for forming the electrodes and the front and rear substrates are attached and sealed using frit. Accordingly, the conventional PDP is complex to fabricate and large in the size of the discharge cell. Therefore, it is difficult to implement high resolution in a small-sized display device using the conventional PDP. Accordingly, there is required a new display device capable of implementing high resolution while being simple and efficient in structure and convenient to fabricate.

### SUMMARY OF THE INVENTION

Aspects of the present invention provide a display device capable of implementing a high resolution while having a simple and efficient structure and a method of fabricating the same.

According to an aspect of the present invention, there is provided a display device including: a substrate; a silicon member attached to the substrate, the silicon member having a groove formed on at least a portion of an inner surface of the silicon member and forming a light-emitting space in cooperation with the substrate; an anode electrode disposed on the substrate; an electron emission member disposed on at least a portion of the groove; and a phosphor layer disposed in the light-emitting space.

While not required in all aspects, the substrate may include glass. The silicon member may include monocrystalline silicon. The silicon member may be formed using a silicon on insulator (SOI) wafer. The SOI wafer may include at least two silicon layers and a silicon oxide (SiO₂) layer formed between the silicon layers. The silicon member may be attached to the substrate by anodic bonding.

While not required in all aspects, the anode electrode may include an indium tin oxide (ITO). When the anode electrode is formed to such a length so as to contact another portion of the inner surface of the silicon member where the groove is not formed, the display device may further include an insulating layer formed on at least a part of the other portion to electrically insulate the anode electrode from the silicon member.

While not required in all aspects, the groove may be formed by an etching process using potassium hydroxide (KOH). The groove may be formed by a deep reactive ion etching (DRIE) process. The insulating layer may include a silicon oxide (SiO₂).

While not required in all aspects, the electron emission member may be formed to include one selected from the group consisting of an oxidized porous silicon, a carbon nano-tube, a diamond like carbon, and a nano-wire. The electron emission member may include an emission electrode. The electron emission member may have one end formed in a tip structure.

While not required in all aspects, the phosphor layer may include one selected from the group consisting of a photoluminescent phosphor, a cathodoluminescent emitting phosphor, a quantum dot and a combination thereof. The light-emitting space may contain excitation gas. The excitation gas may include one selected from the group consisting of xenon (Xe), nitrogen (N₂), heavy hydrogen (D₂), carbon dioxide (CO₂), hydrogen (H₂), carbon monoxide (CO), neon (Ne), helium (He), argon (Ar), air of atmospheric pressure, krypton (Kr) and a combination thereof.

According to another aspect of the present invention, there is provided a display device including: a substrate; a silicon member attached to the substrate, the silicon member having a groove formed on at least a portion of an inner surface of the silicon member and forming a light-emitting space in cooperation with the substrate; an oxidized porous silicon layer disposed at least a portion of the groove; an emission electrode disposed on the oxidized porous silicon layer; and excitation gas disposed in the light-emitting space.

While not required in all aspects, the substrate may include glass. The silicon member may include monocrystalline silicon. The silicon member may be formed using a silicon on insulator (SOI) wafer. The SOI wafer may include at least two silicon layers and a silicon oxide (SiO₂) layer formed between the silicon layers. The silicon member may be attached to the substrate by anodic bonding.

While not required in all aspects, the excitation gas may include one selected from the group consisting of xenon (Xe), nitrogen (N₂), heavy hydrogen (D₂), carbon dioxide (CO₂), hydrogen (H₂), carbon monoxide (CO), neon (Ne), helium (He), argon (Ar), air of atmospheric pressure, krypton (Kr) and a combination thereof.

While not required in all aspects, the display device may further include an anode electrode disposed on an inner surface of the substrate. The display device may further include a phosphor layer disposed in the light-emitting space. The display device may further include a phosphor layer disposed on an outer surface of the substrate to which the silicon member is not attached. The phosphor layer may include one selected from the group consisting of a photoluminescent phosphor, a cathodoluminescent emitting phosphor, a quantum dot and a combination thereof.

According to another aspect of the present invention, there is provided a method of fabricating a display device, the method including: preparing a substrate; forming a groove at an inner surface of a silicon wafer to form a silicon member; forming an electron emission means on at least a portion of the groove; and bonding the substrate and the silicon member by anodic bonding to form a light-emitting space.

While not required in all aspects, the silicon wafer may be an SOI wafer. The groove may be formed by an etching process using potassium hydroxide (KOH). The groove may be formed by a DRIE process.

While not required in all aspects, the anodic bonding process may be performed in a vacuum state. The anodic bonding process may be performed in a place containing excitation gas of a given pressure. The anodic bonding process may be performed in a place containing air of atmospheric pressure.

While not required in all aspects, the method may further include forming an anode electrode on an inner surface of the substrate. Although not required in all aspects, the method may further include forming a phosphor layer in the light-emitting space.

Additional aspects and/or advantages of the invention will be set forth in part in the description which follows and, in part, will be obvious from the description, or may be learned by practice of the invention.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1 is an exploded perspective view of a conventional alternating current (AC) plasma display panel (PDP);

FIG. 2 is a cutaway perspective view of a display device according to an embodiment of the present invention;

FIG. 3 is a sectional view taken along line III-III of FIG. 2;

FIG. 4 is a right side view of the display device illustrated in FIG. 2;

FIGS. 5 through 8 are sectional views illustrating a process of forming a silicon member according to an embodiment of the present invention;

FIG. 9 is a cutaway perspective view of a display device according to another embodiment of the present invention;

FIG. 10 is a sectional view taken along line X-X of FIG. 9; and

FIGS. 11 through 14 are sectional views illustrating a process of forming a silicon member according to another embodiment of the present invention.

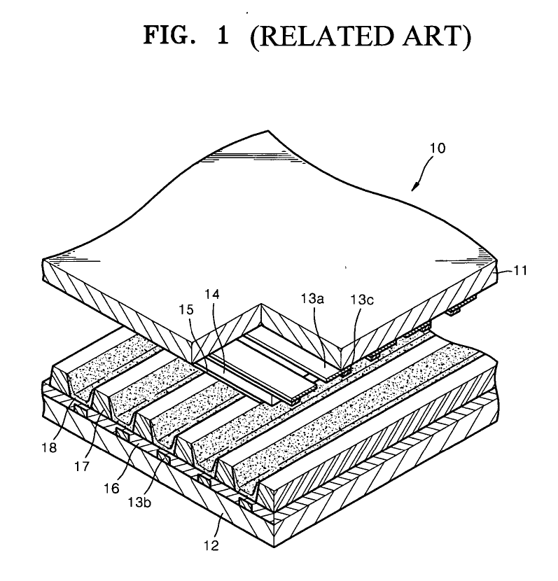

### DETAILED DESCRIPTION OF THE EMBODIMENTS

Reference will now be made in detail to the present embodiments of the present invention, examples of which are illustrated in the accompanying drawings, wherein like reference numerals refer to the like elements throughout. The embodiments are described below in order to explain the present invention by referring to the figures.

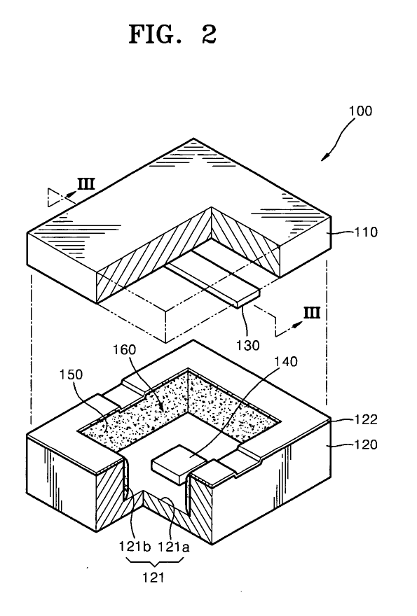

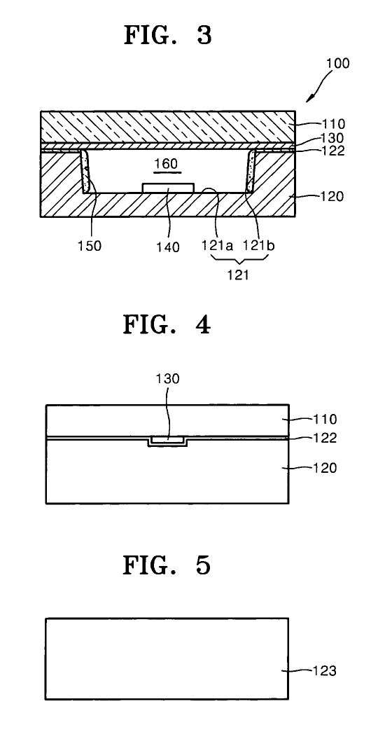

FIG. 2 is a cutaway perspective view of a display device according to an embodiment of the present invention, FIG. 3 is a sectional view taken along line III-III of FIG. 2, and FIG. 4 is a right side view of the display device illustrated in FIG. 2. Referring to FIGS. 2 through 4, a display device 100 includes a substrate 110, a silicon member 120, an anode electrode 130, an electron emission member 140, and a phosphor layer 150.

The substrate 110 is formed of transparent glass, and thus visible rays are transmitted through the substrate 110. The silicon member 120 is formed of monocrystalline silicon, and thus a driving circuit can be directly disposed on the silicon member 120. Although the silicon member 120 is described as formed using a single silicon wafer, the present invention is not limited to this structure. For example, the silicon member 120 may be formed using silicon on insulator (SOI).

The silicon member 120 has the shape of a cuboid with a groove 121 formed on its inner surface. The groove 121 includes a base portion 121a and a side portion 121b, and serves to form a light-emitting space 160 in cooperation with the substrate 110 when the display device 100 is completely assembled. Although the groove 121 is described as having the shape of a cuboid, the present invention is not limited to this structure. That is, the groove 121 may have various shapes such as a cylinder, a polygon, and a hemisphere. Accordingly, the light-emitting space 160 may have various shapes.

An insulating layer 122 is formed on a portion of the inner surface of the silicon member 120 where the groove 121 is not formed. The insulating layer 122 is formed for electrical insulation between the silicon member 120 and the anode electrode 130, and may be formed using a material such as a silicon oxide (SiO₂) and a lead oxide (PbO).

Although the insulating layer 122 is described as on all of the portion of the inner surface of the silicon member 120 where the groove 121 is not formed, the present invention is not limited to this structure. That is, the insulating layer 122 may be formed to a minimum area necessary for the electrical insulation between the silicon member 120 and the anode electrode 130. Also, when the anode electrode 130 is formed only on the center of the substrate 110 that does not contact the silicon member 120, the insulation layer 122 may not be needed at all.

The anode electrode 130 is disposed on an inner surface of the substrate 110, and is formed in a stripe pattern. Although the anode electrode 130 is described as being formed in the stripe pattern, the present invention is not limited to this structure. That is, the anode electrode 130 may be formed on the center of the substrate 110 so that it does not contact the silicon member 120. In this case, the anode electrode 130 may be formed in various shapes, such as a rectangular shape or a circular shape. In this case, a connection hole for connecting the anode electrode 130 to an external power source may be formed in the substrate 110.

The anode electrode 130 may be a transparent electrode formed using an indium tin oxide (ITO). Although the anode electrode 130 is described as being formed using an ITO electrode, the present invention is not limited to this structure. That is, the anode electrode 130 may be formed using other materials, such as silver (Ag), copper (Cu), and aluminum (Al). In order to increase the transmittance of visible light, the anode electrode 130 is preferably formed of a transparent material.

The electron emission member 140 has a section with the shape of a quadrangle, and is disposed at the base portion 121a of the groove 121. The electron emission member 140 emits electrons toward the light-emitting space 160 when the display device 100 operates. In the present embodiment, the electron emission member 140 is formed of an oxidized porous silicon layer. The oxidized porous silicon layer accelerates electrons, and may be formed of oxidized porous silicon or oxidized porous amorphous silicon.

Although the electron emission member 140 is described as being formed of the oxidized porous silicon layer, the present invention is not limited to this structure. That is, the electron emission member 140 may be formed to include one selected from the group consisting of oxidized porous silicon, a carbon nano-tube, a diamond like carbon (DLC), and a nano-wire. That is, the electron emission member may be any one of electron emission sources known in the art used in display devices such as a field emission display (FED), a surface-conduction electron-emitter display (SED), and a metal insulator metal display (MIMD). Although the electron emission means 140 is described as being formed of only an oxidized porous silicon layer having a section with the shape of a quadrangle, the present invention is not limited to this structure. That is, the electron emission member 140 may further include an emission electrode disposed on the oxidized porous silicon layer, and may have a tipped end for easily emitting electrons.

The phosphor layer 150 is formed on the side portion 121b of the groove 121 and an inner surface of the insulating layer 122. The phosphor layer 150 may be formed using various kinds of phosphors. In the present embodiment, the phosphor layer 150 is formed using a photoluminescent phosphor. The photoluminescent phosphor has an element that emits visible light when receiving ultraviolet rays. For example, a red phosphor layer emitting red visible light includes a phosphor such as Y(V,P)O₄:Eu, a green phosphor layer emitting green visible light includes a phosphor such as Zn₂SiO₄:Mn, and a blue phosphor layer emitting blue visible light includes a phosphor such as BAM:Eu.

Although the phosphor layer 150 is described as being formed of a photoluminescent phosphor, the present invention is not limited to this structure. For example, the phosphor layer 150 may be formed using a cathodoluminescent phosphor or a quantum dot. That is, the phosphor layer 150 may be formed using one selected from the group consisting of a photoluminescent phosphor, a cathodoluminescent phosphor, and a quantum dot. Also, the display device 100 may not include the phosphor layer. In this case, excitation gas is contained in the light-emitting space, and the emitting operation is performed using only visible light emitted by the excitation gas. In this case, when a discharge occurs in the light-emitting space 160, more visible light is emitted by the excitation gas.

Although the phosphor layer 150 is described as being formed on the side portion 121b of the groove 121 and the inner surface of the insulating layer 122, the present invention is not limited to this structure. That is, the phosphor layer 150 may be disposed at any place so long as it can receive electrons or ultraviolet rays to emit light. That is, the phosphor layer 150 may be disposed not only at any place in the light-emitting space 160, but also on an outer surface of the substrate 110 to which the silicon member 120 is not attached. The case where the phosphor layer 150 is disposed on the outer surface of the substrate 110 may be applied to a case where the light-emitting space 160 is hermetically filled with excitation gas emitting long-wavelength ultraviolet rays, such as nitrogen (N₂).

As described above, after the anode electrode 130 is disposed on the substrate 110 and the groove 121, the insulating layer 122, the electron emission member 140 and the phosphor layer 150 are formed on the silicon member 120, the silicon member 120 is attached to the substrate 110, thereby forming the display device 100 with the light-emitting space 160. The silicon member 120 may be attached by anodic bonding to the substrate 110. In the anodic bonding process, the light-emitting space 160 is hermetically filled with excitation gas mixed with xenon (Xe), for example. The excitation gas may include one selected from the group consisting of xenon (Xe), nitrogen (N₂), heavy hydrogen (D₂), carbon dioxide (CO₂), hydrogen (H₂), carbon monoxide (CO), neon (Ne), helium (He), argon (Ar), air of atmospheric pressure, krypton (Kr) and a combination thereof. Although the display device 100 is described as including excitation gas, the present invention is not limited to this structure. That is, the light-emitting space 160 may maintain a vacuum state without containing the excitation gas. This case corresponds to a case where a phosphor layer containing a cathodoluminescent phosphor or a quantum dot is formed in the light-emitting space 160 and electrons emitted from the electron emission member 140 are directly irradiated onto the phosphor layer to emit visible light.

A method of fabricating the display device 100 will now be described in detail. First, an anode electrode 130 is formed on an inner surface of a glass substrate 110 by a printing process. Next, the silicon member 120 is formed by a process which will now be described in detail with reference to FIGS. 5 through 8.

FIGS. 5 through 8 are sectional views illustrating a process of forming the silicon member 120 according to an embodiment of the present invention. The silicon member 120 includes a groove 121 having a base portion 121a and a side portion 121b. The groove 121 may be formed by wet etching or dry etching. In the present embodiment, the groove 121 is formed by wet etching. As illustrated in FIGS. 5 and 6, a silicon wafer 123 with a given size is etched using a potassium hydroxide (KOH) solution, thereby forming the silicon member 120 with the groove 121 formed therein. At this point, a silicon layer 120a with a given height is formed on the base portion 121a of the groove 121.

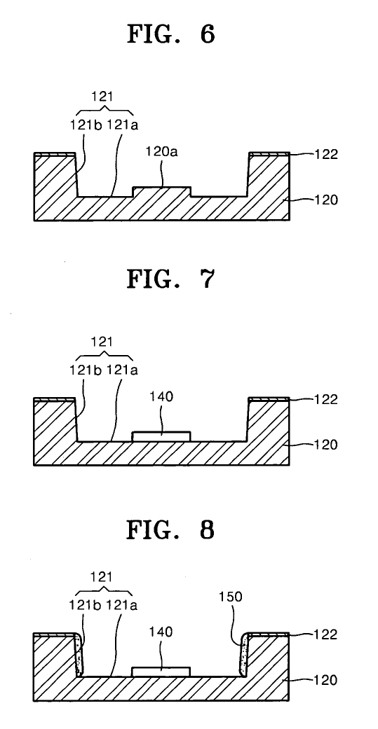

Thereafter, as illustrated in FIG. 7, the silicon layer 120a is changed into an oxidized porous silicon layer to constitute an electron emission member 140, and an insulating layer 122 is formed on an inner surface of the silicon member 120 at a portion where the groove 121 is not formed. The insulating layer 122 may be formed of a silicon oxide (SiO₂) by a printing process.

The oxidized porous silicon layer may be formed by the following method. A suitable current density is applied to the silicon layer 120a, and the silicon layer 120a is changed into a porous state by anodizing it using a solution mixture of hydrogen fluoride (HF) and ethanol. Thereafter, the anodized silicon layer is changed into an oxidized porous silicon layer by oxidizing it by electrochemical oxidation.

In the present embodiment, in order to form the silicon layer 120a, a portion of the base portion 121a is less etched than the other portions during the forming of the groove 121. However, the present invention is not limited to this method. That is, the silicon layer 120a may be separately formed on the base portion of the groove after a groove with a flat base portion is formed, for example, by a printing process. Thereafter, as illustrated in FIG. 8, a phosphor layer 150 is coated on the side portion 121b of the groove 121 and an inner surface of the insulating layer 122.

Thereafter, the silicon member 120 is attached to the substrate 110. The silicon member 120 is joined to the substrate 110 in a chamber containing excitation gas of a given pressure. At this point, the silicon member 120 is attached to the substrate 110 by anodic bonding. In the anodic bonding process, after the silicon member 120 is brought into contact with the substrate 110, the silicon member 120 and the substrate 110 are joined together by chemical reaction. This chemical reaction may be generated by a high voltage applied at a high temperature of about 450° C.

When using the anodic bonding process, the silicon member 120 and the substrate 110 can be joined together while maintaining the insulating layer 122. In this case, destruction of the insulating layer 122 can be prevented while maintaining the gastightness of the light-emitting space 160. Accordingly, as illustrated in FIG. 4, the insulating state between the silicon member 120 and the anode electrode 130 can be maintained even after the silicon member 120 and the substrate 110 are joined together.

In the present embodiment, the anodic bonding process for attaching the silicon member 120 to the substrate 110 is performed in a chamber containing excitation gas of a given pressure and thus the excitation gas is placed into the light-emitting space 160. However, the present invention is not limited thereto. That is, the anodic bonding process may also be performed in a general atmospheric environment. In addition, a discharge hole may be formed in the substrate 110 after the anodic bonding process to discharge air of the light-emitting space 160, and then suitable excitation gas may be injected through the discharge hole into the light-emitting space 160.

An operation of the display device 100 will now be described in detail. First, voltages are applied from an external power source to the silicon member 120 and the anode electrode 130, respectively. At this time, the voltage V1 applied to the silicon member 120 is lower than the voltage V2 applied to the anode electrode 130.

Due to the applied voltage, a current flows though the silicon member 120 functioning as a cathode electrode. The reason for this is that the light-emitting space 160 is much higher in resistance than the silicon member 120. According to the current flow, electrons flow from the silicon member 120 into the electron emission member 140. The electrons having flowed into the electron emission member 140 are accelerated by the oxidized porous silicon layer and then emitted into the light-emitting space 160. The electrons emitted into the light-emitting space 160 excite the excitation gas. At this time, depending on the level of the applied voltage, a discharge may occur in the light-emitting space 160. This excited gas is stabilized to emit ultraviolet rays. The emitted ultraviolet rays excite the phosphor layer 150 formed of a photoluminescent phosphor. Accordingly, visible light is generated and outputted through the substrate 110, thereby creating an image.

In the present embodiment, the excitation gas is excited by the electrons emitted from the electron emission member 140, the excited gas is stabilized to generate ultraviolet rays, and the generated ultraviolet rays excite the phosphor layer to generate the visible light. However, the present invention is not limited to this structure. That is, as described above, the phosphor layer may be formed using a cathodoluminescent phosphor or a quantum dot. In this case, irrespective of the excitation process of the excitation gas, visible light may be generated merely by the direct collision between the emitted electrons and the phosphor layer.

As described above, the display device 100 is simple in structure and can be easily fabricated using a minute silicon process. Therefore, the display device 100 can be miniaturized and thus a minute light-emitting cell can be implemented. Accordingly, it is possible to implement the display device with high resolution. Also, when the display device 100 is arranged in a tile pattern, a display device with a desired size can be implemented. Accordingly, it is possible to easily implement a large-screen display device. Also, since the silicon member 120 is formed using monocrystalline silicon, the driving circuit can be directly formed on the silicon member 120, thereby reducing the required space and cost.

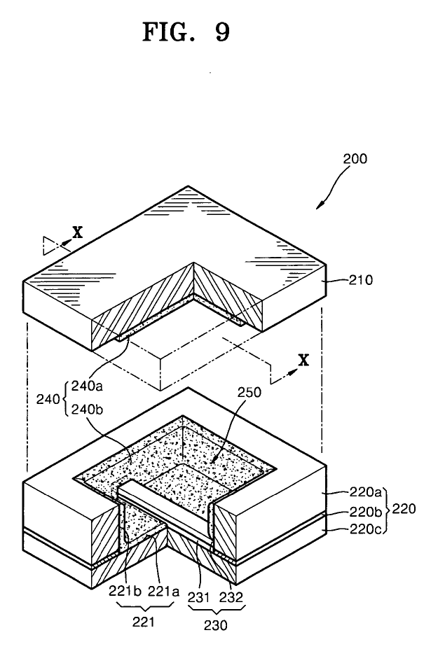

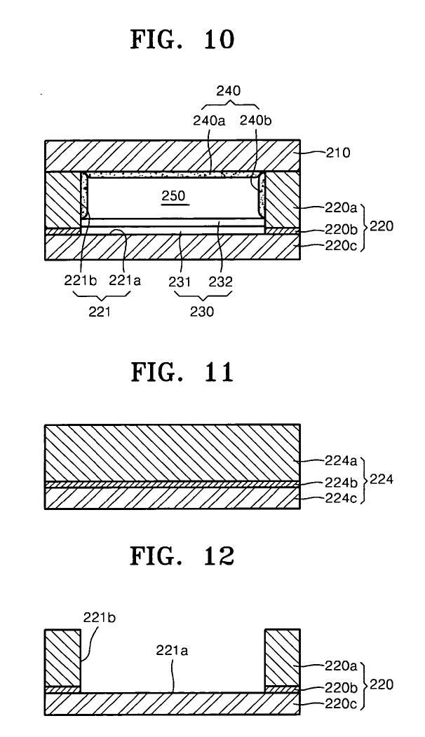

Hereinafter, another embodiment of the present invention will be described in detail with reference to FIGS. 9 through 13. FIG. 9 is a cutaway perspective view of a display device according to another embodiment of the present invention, and FIG. 10 is a sectional view taken along line X-X of FIG. 9. Referring to FIGS. 9 and 10, a display device 200 includes a substrate 210, a silicon member 220, an electron emission member 230, and a phosphor layer 240. The substrate 210 is formed of transparent glass such that visible rays are transmitted through the substrate 210. The silicon member 220 is formed of monocrystalline silicon, and thus a driving circuit can be directly disposed on the silicon member 220.

In the present embodiment, the silicon member 220 is formed using an SOI wafer. The SOI wafer includes two silicon layers and a silicon oxide layer formed therebetween. Accordingly, the silicon member 220 includes a first silicon layer 220a, a second silicon layer 220c, and a silicon oxide layer 220b formed between the first and second silicon layers 220a and 220c. The silicon oxide layer 220b has an electrically insulative property, and functions to easily adjust an etching depth during an etching process.

The silicon member 220 has the shape of a cuboid with a groove 221 formed on its inner surface. The groove 221 includes a base portion 221a and a side portion 221b. The groove 221 is formed such that the base portion 221a becomes an inner surface of the second silicon layer 220c and the side portion 221b becomes an inner surface of a portion remaining after the first silicon layer 220a and the silicon oxide layer 220b are etched. The groove 221 serves to form a light-emitting space 250 in cooperation with the substrate 210 when the display 200 is completely assembled.

The electron emission member 230 includes an oxidized porous silicon layer 231 and an emission electrode 232. The oxidized porous silicon layer 231 is disposed on an inner surface of the second silicon layer 220c, i.e., the base portion 221a of the groove 221, and is formed in a stripe shape such that its both ends contact the side portion 221b of the groove 221. The oxidized porous silicon layer 231 may be formed of oxidized porous polycrystalline silicon or oxidized porous amorphous silicon.

While the present embodiment illustrates that the oxidized porous silicon layer 231 has both ends formed in contact with the side portion 221b of the groove 221, the present invention is not limited to this. In other words, the oxidized porous silicon layer 231 does not need to contact the side portion 221b of the groove 221. However, it is preferable that both ends of the oxidized porous silicon layer 231 be in contact with the side portion 221b of the groove 221 so as to make it easy to form the emission electrode 232.

The emission electrode 232 is formed on the oxidized porous silicon layer 231 in a stripe pattern. The emission electrode 232 is formed in a mesh structure such that electrons accelerated by the oxidized porous silicon layer 231 are easily emitted. The emission electrode 232 is designed such that both ends thereof are in contact with the first silicon layer 220a, so that the emission electrode 232 is electrically connected with the first silicon layer 220a.

The phosphor layer 240 is formed on a predetermined portion of the emission space 250 except for some of the portions where the oxidized porous silicon layer 231 and the emission electrode 232 are positioned. The phosphor layer 240 is divided into a first phosphor layer 240a formed on the substrate 210 and a second phosphor layer 240b formed in the groove 221 of the silicon member 220. The first phosphor layer 240a is formed on a selected portion of an inner surface of the substrate 210 where the emission space 250 is positioned. Since the selected portion corresponds to a position to which the electrons emitted from the emission electrode 232 directly collide, the first phosphor layer 240a is made of a cathodoluminescent phosphor. The second phosphor layer 240b is formed on a predetermined portion of the groove 221 of the silicon member 220 except for some of the portions where the oxidized porous silicon layer 231 and the emission electrode 232 are positioned. The second phosphor layer 240b is made of a photo-luminescent phosphor that receives ultraviolet rays to emit visible rays.

As above, after the first phosphor layer 240a is disposed on the substrate 210 and the groove 221, the electron emission member 230 and the second phosphor layer 240b are formed on the silicon member 220, the silicon member 220 is attached to the substrate 210, thereby forming the display device 200 with the light-emitting space 250. The silicon member 220 may be attached by anodic bonding to the substrate 210. In the anodic bonding process, the light-emitting space 250 is hermetically filled with excitation gas mixed with xenon (Xe), for example. The excitation gas may include one selected from the group consisting of xenon (Xe), nitrogen (N₂), heavy hydrogen (D₂), carbon dioxide (CO₂), hydrogen (H₂), carbon monoxide (CO), helium (He), argon (Ar), air of atmospheric pressure, krypton (Kr) and combinations thereof.

A method of fabricating the display device 200 will now be described in detail. First, a first phosphor layer 240a is formed on an inner surface of a glass substrate 210 by a printing process.

Next, a process of forming the silicon member 220 is carried out which will now be described in detail with reference to FIGS. 11 through 14. FIGS. 11 through 14 are sectional views illustrating a process of forming the silicon member 220 according to another embodiment of the present invention. The silicon member 220 includes a groove 221. In the present embodiment, the groove 221 is formed by a dry etching process, such as a DRIE process.

As illustrated in FIGS. 11 and 12, the silicon member 220 is formed using an SOI wafer 224. The SOI wafer 224 includes a first silicon layer 224a, a second silicon layer 224c, and a silicon oxide layer 224b formed between the layers 224a and 224c. The SOI wafer 224 is etched by a DRIE process. The DRIE process can form a more vertical etching surface than the wet etching process using KOH. Therefore, the discharge space defined by the groove 221 can be formed larger, resulting in a larger light-emitting space 250. Also, since the silicon member 220 is formed by etching the SOI wafer 224, the etching depth can be easily adjusted. That is, the silicon oxide layer 224b functions to prevent the second silicon layer 224c from being etched during the DRIE process.

Accordingly, the silicon member 220 includes the first silicon layer 220a with the shape of a quadrangle tube, the silicon oxide layer 220b with the shape of the quadrangle tube, and the second silicon layer 220c with the shape of a plate. Consequently, the silicon member 220 has the shape with the groove 221. Accordingly, the base portion 221a of the groove 221 becomes an inner surface of the second silicon layer 220c, and the side portion 221b becomes an inner surface of a portion remaining after the first silicon layer 220a and the silicon oxide layer 220b are etched.

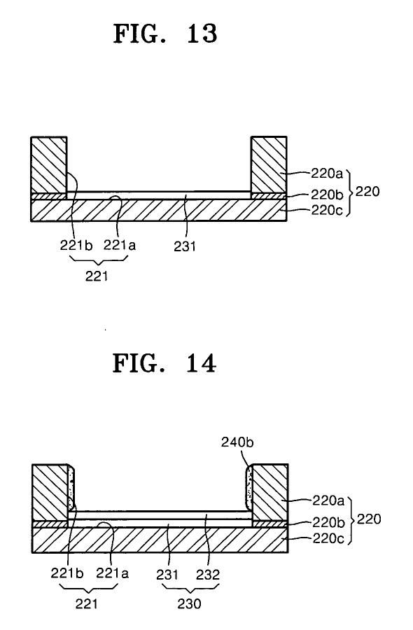

Thereafter, as illustrated in FIG. 13, an oxidized porous silicon layer 231 is formed on an inner surface of the second silicon layer 220c (i.e., the base portion 221a of the groove 221). In the present embodiment, the oxidized porous silicon layer 231 is formed by depositing a silicon layer with a given thickness on the second silicon layer 220c and changing the deposited silicon layer into an oxidized porous state by anodization or electrochemical oxidation. Thereafter, as illustrated in FIG. 14, an emission electrode 232 is formed on the oxidized porous silicon layer 231 such that both ends of the emission electrode 232 contact the first silicon layer 220a. Thereafter, a photoluminescent phosphor is coated on the groove 221 of the silicon member 220, thereby forming a second phosphor layer 240b. Thereafter, the silicon member 220 is attached to the substrate 210. The silicon member 220 is joined to the substrate 210 in a chamber containing excitation gas of a given pressure. At this point, the silicon member 220 is attached to the substrate 210 by anodic bonding.

An operation of the display device 200 will now be described in detail. First, voltages are applied from an external power source to the first silicon layer 220a and the second silicon layer 220c, respectively. At this time, the voltage V3 applied to the first silicon layer 220a is higher than the voltage V4 applied to the second silicon layer 220c. At this time, since the first silicon layer 220a is electrically connected to the emission electrode 232, the emission electrode 232 has a voltage of V3.

Due to the applied voltage, electrons flow from the second silicon layer 220c into the oxidized porous silicon layer 231. The electrons having flowed into the oxidized porous silicon layer 231 are accelerated and then emitted into the light-emitting space 250 through the emission electrode 232. The electrons emitted into the light-emitting space 250 directly collide against the first phosphor layer 240a, which is formed of a cathodoluminescent phosphor, to generate visible light, or the emitted electrons excite the excitation gas to generate ultraviolet rays. The generated ultraviolet rays excite the first phosphor layer 240a formed of a photoluminescent phosphor. The energy level of the excited phosphor is lowered to emit visible light. The visible light emitted from the first phosphor layer 240a and the second phosphor layer 240b is outputted through the substrate 210, thereby creating an image.

In the present embodiment, the substrate 210 does not include the anode electrode. However, the present invention is not limited to this structure. That is, the substrate may further include the anode electrode. In this case, a voltage higher than a voltage applied to the emission electrode may be applied to the anode electrode so as to attract the emitted electrons.

As above, the display device 200 is simple in structure and can be miniaturized. Accordingly, it is possible to implement the display device with high resolution. Also, when the display device 200 is arranged in a tile pattern, a display device with a desired size can be implemented. Accordingly, it is possible to easily implement a large-screen display device. Also, since the silicon member 220 is formed using monocrystalline silicon, the driving circuit can be directly formed on the silicon member 220, thereby reducing the required space and cost. Also, since the display device does not have to have the anode electrode, no separate insulation needs to be formed. Accordingly, the fabrication process can be simplified and the fabrication cost can be reduced. Also, since the silicon member 220 is formed using the SOI wafer 224, the etching depth for the groove 221 can be easily adjusted to implement the precise structure. Therefore, it is possible to reduce the defective percentage and increase the fabrication speed.

As described above, the display device is simple in structure and can be easily fabricated using a minute silicon process. Therefore, the display device can be miniaturized and thus the minute light-emitting cell can be implemented. Accordingly, it is possible to implement a high resolution display even to a small-sized display device. Also, when the display device is arranged in a tile pattern, the display area can be adjusted to a desired value. Accordingly, it is possible to easily implement a large-sized display device. Also, since the silicon member is formed using monocrystalline silicon, the driving circuit can be directly formed on the silicon member, thereby reducing the required space and cost. Also, since the silicon member is formed using the SOI wafer, the etching depth for the groove can be easily adjusted to implement the precise structure. Therefore, it is possible to reduce the defective percentage and increase the fabrication speed.

Although a few embodiments of the present invention have been shown and described, it would be appreciated by those skilled in the art that changes may be made in this embodiment without departing from the principles and spirit of the invention, the scope of which is defined in the claims and their equivalents.

What is claimed is:

1. A display device comprising:
a substrate;
a silicon member attached to the substrate, the silicon member having a groove formed on at least a portion of an inner surface of the silicon member and forming a light-emitting space in cooperation with the substrate;
an anode electrode disposed on the substrate;
an electron emission member disposed on at least a portion of the groove; and
a phosphor layer disposed in the light-emitting space.

2. The display device of claim 1, wherein the substrate includes glass.

3. The display device of claim 1, wherein the silicon member includes monocrystalline silicon.

4. The display device of claim 3, further comprising a driving circuit formed on the silicon member.

5. The display device of claim 1, wherein the silicon member is formed using an silicon on insulator (SOI) wafer.

6. The display device of claim 5, wherein the SOI wafer includes at least two silicon layers and a silicon oxide (SiO₂) layer formed between the silicon layers.

7. The display device of claim 1, wherein the silicon member is attached to the substrate by anodic bonding.

8. The display device of claim 1, wherein the anode electrode includes an indium tin oxide (ITO).

9. The display device of claim 1, wherein the anode electrode is formed only on a center of the substrate and does not contact the silicon member.

10. The display device of claim 1, wherein the anode electrode has a length to contact another portion of the inner surface of the silicon member where the groove is not formed and the display device further includes an insulating layer formed on at least a part of the another portion to electrically insulate the anode electrode from the silicon member.

11. The display device of claim 10, wherein the insulating layer includes a silicon oxide (SiO₂).

12. The display device of claim 1, wherein the electron emission member comprises one selected from the group consisting of an oxidized porous silicon, oxidized porous amorphous silicon, a carbon nano-tube, a diamond like carbon, and a nano-wire.

13. The display device of claim 1, wherein the electron emission member includes an emission electrode.

14. The display device of claim 1, wherein the electron emission member has one end formed in a tip structure.

15. The display device of claim 1, wherein the phosphor layer includes one selected from the group consisting of a photoluminescent phosphor, a cathodoluminescent emitting phosphor, and a quantum dot.

16. The display device of claim 1, wherein the light-emitting space contains excitation gas.

17. The display device of claim 16, wherein the excitation gas includes one selected from the group consisting of xenon (Xe), nitrogen (N₂), heavy hydrogen (D₂), carbon dioxide (CO₂), hydrogen (H₂), carbon monoxide (CO), neon (Ne), helium (He), argon (Ar), air of atmospheric pressure, krypton (Kr) and combinations thereof.

18. A flat panel display screen comprising the display device of claim 1 arranged in a tile pattern to form a display screen of a certain size.

19. A display device comprising:
a substrate;
a silicon member attached to the substrate, the silicon member having a groove formed on at least a portion of an inner surface of the silicon member and forming a light-emitting space in cooperation with the substrate;
an oxidized porous silicon layer disposed at least a portion of the groove;
an emission electrode disposed on the oxidized porous silicon layer; and
excitation gas disposed in the light-emitting space.

20. The display device of claim 19, wherein the substrate includes glass.

21. The display device of claim 19, wherein the silicon member includes monocrystalline silicon.

22. The display device of claim 21, further comprising a driving circuit formed on the silicon member.

23. The display device of claim 19, wherein the silicon member is formed using a silicon on insulator (SOI) wafer.

24. The display device of claim 23, wherein the SOI wafer includes at least two silicon layers and a silicon oxide (SiO₂) layer formed between the silicon layers.

25. The display device of claim 24, wherein the oxidized porous silicon layer is disposed on only one of the silicon layers and the emission electrode disposed on the oxidized porous silicon layer contacts only the other silicon layer of the two silicon layers.

26. The display device of claim 19, wherein the silicon member is attached to the substrate by anodic bonding.

27. The display device of claim 19, wherein the excitation gas includes one selected from the group consisting of xenon (Xe), nitrogen (N₂), heavy hydrogen (D₂), carbon dioxide (CO₂), hydrogen (H₂), carbon monoxide (CO), neon (Ne), helium (He), argon (Ar), air of atmospheric pressure, krypton (Kr) and combinations thereof.

28. The display device of claim 19, further comprising an anode electrode disposed on an inner surface of the substrate.

29. The display device of claim 28, wherein the anode electrode includes indium tin oxide (ITO).

30. The display device of claim 19, further comprising a phosphor layer disposed in the light-emitting space.

31. The display device of claim 30, wherein the phosphor layer includes one selected from the group consisting of a photoluminescent phosphor, a cathodoluminescent emitting phosphor, and a quantum dot.

32. The display device of claim 19, further comprising a phosphor layer disposed on an outer surface of the substrate to which the silicon member is not attached.

33. A flat panel display screen comprising the display device of claim 19 arranged in a tile pattern to form a display screen of a certain size.

34. A display device comprising:
a first substrate to transmit light;
a second substrate semiconductor attached to the first substrate, the second substrate having a groove formed on at least a portion of a surface of and forming a light-emitting space in cooperation with the first substrate;
an oxidized porous silicon layer disposed on at least a portion of the groove;
an electron emission electrode disposed on the oxidized porous silicon layer; and
excitation gas disposed in the light-emitting space.
